(19) United States
(12) Patent Application Publication
(10) Pub. No.: US 2025/0135083 A1
(43) Pub. Date: May 1, 2025

(54) PRESSURE SENSORS FOR OPHTHALMIC SURGICAL CONSOLES AND CASSETTES

(71) Applicant: Alcon Inc., Fribourg (CH)

(72) Inventor: Vincent A. BAXTER, Temecula, CA (US)

(21) Appl. No.: 18/888,005

(22) Filed: Sep. 17, 2024

Related U.S. Application Data
(60) Provisional application No. 63/594,902, filed on Oct. 31, 2023.

(51) Int. Cl. A61M 1/00

(52) U.S. Cl. CPC A61M 1/72; A61M 2205/3348

(57) ABSTRACT

A sensor assembly for sensing fluid pressure in a surgical cassette is provided. The sensor assembly includes a tubing having a first portion and a second portion, a follower assembly operable to engage with a retaining ring of the surgical cassette coupled to the first portion, a collar on the second portion of the tubing operable to fit into a bearing of a pump head of a fluid pump assembly within a surgical console operable to receive the surgical cassette, and a biasing element between the follower assembly and the collar operable to apply a biasing force to the follower assembly to engage the follower with the surgical cassette. The follower assembly includes a follower coupled to a cup and a sensor component.

### CROSS-REFERENCE TO RELATED APPLICATIONS

This application claims priority to U.S. Provisional Application No. 63/594,902, filed on Oct. 31, 2023, which is hereby incorporated by reference in its entirety.

### INTRODUCTION

This section provides background information to facilitate a better understanding of the various aspects of the disclosure. It should be understood that the statements in this section of this document are to be read in this light, and not as admissions of prior art.

Ophthalmic surgical procedures are often classified as anterior segment surgical procedures, posterior segment procedures, or combined anterior segment and posterior segment procedures (i.e., “combined procedures”). The anterior segment refers to the front-most region of the eye, and includes the cornea, iris, and lens. Thus, anterior segment surgical procedures typically include surgeries performed on the iris and/or lens, such as cataract surgery. The posterior segment refers to the back-most region of the eye that includes the anterior hyaloid membrane and the optical structures behind it, such as the vitreous humor, the retina, the choroid, and the optic nerve. Posterior segment surgical procedures typically include retinal and vitreoretinal surgeries. In certain cases, a patient may have pathologies of the eye requiring both anterior and posterior procedures; in such cases, a combined procedure may be performed.

During anterior and/or posterior segment surgery, tissue fragments and other materials may be aspirated or suctioned out of the eye through, e.g., a hollow needle or cannula. Also, during the procedure, an irrigating or infusion fluid may be pumped into the eye to maintain an intraocular pressure (IOP) and prevent collapse of the eye. A surgical cassette having one or more peristaltic and/or venturi pumps and one or more valve assemblies may be operably coupled with a fluidics module of a surgical console and used to facilitate the aspiration/suction and irrigation/infusion functionalities described above. In general, the one or more valve assemblies of the surgical cassette are operable to control the application of pressure and vacuum generated by the one or more peristaltic pumps during the surgical procedure.

However, conventional pressure sensing devices in surgical consoles that interact with the surgical cassettes are generally assembled rigidly to a module structure and sense deflection of an element on the cassettes. These sensors measure microns or sub-microns of motion on the element. As such, motion, flexing, expansion, and contraction will show up as perceived pressure changes due to the relative change in position between the sensor element and the sensed element.

Therefore, there is a need for improved sensor assemblies that address at least some of the drawbacks caused by conventional pressure devices.

### SUMMARY

This summary is provided to introduce a selection of concepts that are further described below in the Detailed Description. This summary is not intended to identify key or essential features of the claimed subject matter, nor is it to be used as an aid in limiting the scope of the claimed subject matter.

In one embodiment, a sensor assembly for sensing fluid pressure in a surgical cassette is disclosed herein. The sensor assembly includes a tubing having a first portion and a second portion, a follower assembly operable to engage with a retaining ring of the surgical cassette coupled to the first portion, a collar on the second portion of the tubing operable to fit into a bearing of a pump head of a fluid pump assembly within a surgical console operable to receive the surgical cassette, and a biasing element between the follower assembly and the collar operable to apply a biasing force to the follower assembly to engage the follower with the surgical cassette. The follower assembly includes a follower coupled to a cup and a sensor component.

In another embodiment, a sensor assembly for sensing fluid pressure in a surgical cassette attached to a surgical console is disclosed herein. The sensor assembly includes a tubing having a first portion and a second portion, a follower assembly operable to engage with a retaining ring of the surgical cassette coupled to the first portion, a collar on the second portion of the tubing operable to fit into a bearing of a pump head of a fluid pump assembly within a surgical console operable to receive the surgical cassette, and a biasing element between the follower assembly and the collar operable to apply a biasing force to the follower assembly to engage the follower with the surgical cassette. The follower assembly includes a follower coupled to a cup and a mechanical stop positioned between the follower and the cup. The mechanical stop is disposed at an end of the first portion of the tubing and the mechanical stop includes a first alignment mechanism corresponding to a second alignment mechanism on an inside portion of the cup. The follower assembly additionally includes a strain relief positioned between the mechanical stop and the follower, a sensor component having a printed circuit board assembly (PCBA) with a coil positioned in a center portion of the follower and abutting a portion of the strain relief, and a PCBA support abutting a PCBA spring. The PCBA contacts a portion of the PCBA support and a portion of the cup.

### BRIEF DESCRIPTION OF THE DRAWINGS

A more complete understanding of the subject matter of the present disclosure may be obtained by reference to the following Detailed Description when taken in conjunction with the accompanying Drawings wherein:

FIG. 1A illustrates an example of an ophthalmic surgical system that may be used to perform ophthalmic procedures on an eye according to certain embodiments.

FIG. 1B illustrates an example of subsystems of a console of the ophthalmic surgical system of FIG. 1A, according to certain embodiments.

FIG. 2A illustrates a back side perspective view of a surgical cassette which may be operably coupled to a console of an ophthalmic surgical system according to certain embodiments.

FIG. 2B illustrates a back side elevation view of the surgical cassette of FIG. 2A according to certain embodiments.

FIG. 2C is an exploded perspective view of the surgical cassette of FIGS. 2A-2B illustrating the components comprising each of the pump assemblies disposed in the surgical cassette, according to certain embodiments.

FIG. 2D illustrates a top down plan view of a retainer ring that is disposed within the pump assemblies of the surgical cassette of FIGS. 2A-2C according to certain embodiments.

FIG. 3A illustrates a frontal perspective view of an inductive sensor assembly for sensing fluid pressure in the surgical cassette of FIGS. 2A-2B according to certain embodiments.

FIG. 3B illustrates a back side perspective view of the inductive sensor assembly of FIG. 3A according to certain embodiments.

FIG. 3C illustrates an enlarged view of the inductive sensor assembly of FIGS. 3A-3B according to certain embodiments.

FIG. 3D illustrates a backside perspective view of a mechanical stop of the inductive sensor assembly of FIGS. 3A-3C according to certain embodiments.

FIG. 3E illustrates a front side perspective view of a cup of the inductive sensor assembly of FIGS. 3A-3C according to certain embodiments.

FIG. 4A illustrates a frontal perspective view of an eddy current sensor assembly for sensing fluid pressure in the surgical cassette of FIGS. 2A-2B according to certain embodiments.

FIG. 4B illustrates a back side perspective view of the eddy current sensor assembly of FIG. 4A according to certain embodiments.

FIG. 4C illustrates an enlarged view of the eddy current sensor assembly of FIGS. 4A-4B according to certain embodiments.

FIG. 4D illustrates an exploded view of the eddy current sensor assembly of FIG. 4C according to certain embodiments.

FIG. 5A illustrates a frontal perspective view of an optical sensor assembly for sensing fluid pressure in the surgical cassette of FIGS. 2A-2B according to certain embodiments.

FIG. 5B illustrates a back side perspective view of the optical sensor assembly of FIG. 5A according to certain embodiments.

FIG. 5C illustrates an enlarged view of the optical sensor assembly of FIGS. 5A-5B according to certain embodiments.

FIG. 6A illustrates a frontal perspective view of a fluid pump assembly having the inductive sensor assembly of FIGS. 3A-3C therein for sensing fluid pressure in the surgical cassette of FIGS. 2A-2B according to certain embodiments.

FIG. 6B illustrates an enlarged view of the fluid pump assembly of FIG. 6A according to certain embodiments.

FIG. 7 illustrates a frontal perspective view of two fluid pump assemblies of FIGS. 6A-6B, having two eddy current sensor assemblies of FIGS. 4A-4D, within in a fluidic subsystem of the surgical console of FIGS. 1-1B, and an associated surgical cassette of FIGS. 2A-2B according to certain embodiments.

### DETAILED DESCRIPTION

It is to be understood that the following disclosure provides many different embodiments, or examples, for implementing different features of various embodiments. Specific examples of components and arrangements are described below to simplify the disclosure. These are, of course, merely examples and are not intended to be limiting. The section headings used herein are for organizational purposes and are not to be construed as limiting the subject matter described.

The disclosure relates to a pressure sensor assembly for surgical consoles and surgical cassettes whose purpose is to accurately measure vacuum and/or pressure of a fluid or gas within the surgical cassette by measuring the deflection of a diaphragm in contact with the fluid or gas. The embodiments described herein provide improved stability of fluidic measurements by mechanically decoupling the sensor component from the surgical console while mechanically coupling the sensor component to the surgical cassette at the location of the diaphragm. This reduces unwanted relative motion between the diaphragm and sensor components caused from various phenomena in typical designs, such as, for example, vibration, thermal expansion, component flexing, alignment limitations, and mechanical disturbances.

In certain embodiments, the sensor assembly may have a follower assembly such that sensor components interact with a diaphragm by way of a retaining ring on the surgical cassette mating with a follower of the follower assembly. During clamping of the surgical cassette to a surgical console, the surgical cassette is pulled towards the follower assembly and the retaining ring of the cassette contacts the follower. As the cassette is secured in place, the cassette pushes the follower assembly towards the surgical console. The engagement of the sensor component to the surgical cassette, and the decoupling of the sensor component from the surgical console allows the sensor components to follow the surgical cassette while the surgical cassette shifts, vibrates, expands, or contracts during a surgical procedure.

The sensor components can include optical sensors, inductive sensors, eddy current sensors, mechanical force sensors (via strain gauges), and the like. When the surgical cassette is removed, the sensor component is configured to bottom out and align to a position to be ready for another surgical cassette. This is accomplished using a mechanical stop that is within the follower assembly.

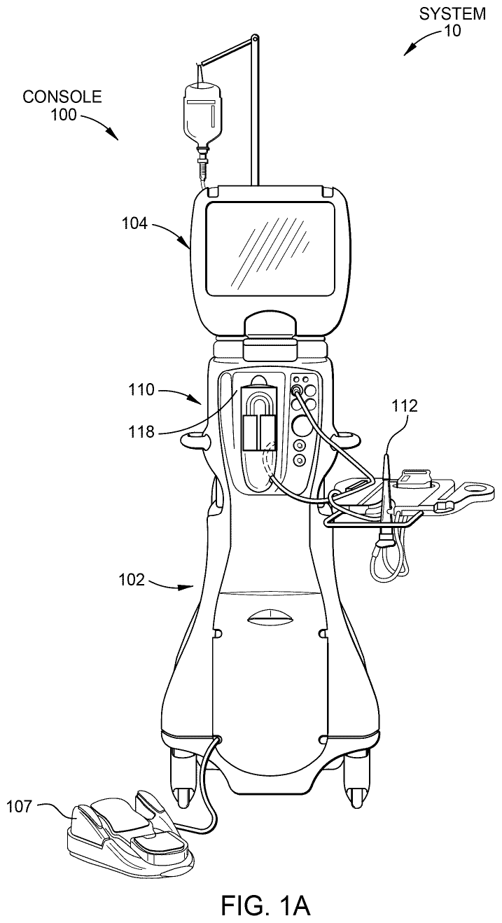

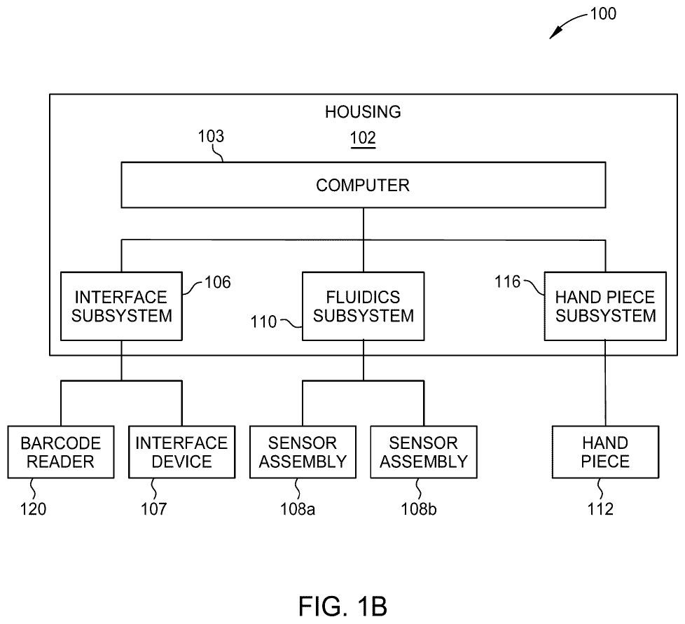

FIG. 1A illustrates an example of an ophthalmic surgical system 10 that may be used to perform ophthalmic procedures on an eye, according to certain embodiments. FIG. 1B is an example of subsystems of console 100 of ophthalmic surgical system 10 of FIG. 1A, according to certain embodiments. FIGS. 1A-1B are described together herein for clarity. In the illustrated embodiments, ophthalmic surgical system 10 includes surgical console 100 having a housing 102, a display screen 104, an interface device 107, a fluidics subsystem 110, face plate 118, and a hand piece 112. Housing 102 accommodates a computer 103 with an associated display screen 104 and one or more subsystems to support the interface device 107 and the hand piece 112. The interface device 107 receives input to the ophthalmic surgical system 10, sends output from the ophthalmic surgical system 10, and/or processes the input and/or output via the interface subsystem 106. In certain embodiments, the interface device 107 can include, without limitation, a foot pedal, manual input device (e.g., a keyboard), a display, and combinations of the same and like. In certain embodiments, a barcode reader 120 may be integrated with the surgical console 100 via the interface subsystem 106. In some embodiments, the barcode reader 120 may be configured to capture an image of a barcode on a surgical cassette attached to the surgical console 100 and map the captured image to an identity or type of the surgical cassette, and/or map the captured image to stored calibration data of the surgical cassette.

In certain embodiments, the hand piece 112 may be any suitable ophthalmic surgical instrument, such as, for example, an ultrasonically-driven phacoemulsification (phaco) hand piece, a laser hand piece, an irrigating cannula, a vitrectomy hand piece, or another suitable surgical hand piece. In various embodiments, the hand piece 112 can include a plurality of hand pieces 112. The hand piece 112 can interact with the ophthalmic surgical system 10 via the hand piece subsystem 116. The fluidics subsystem 110 provides fluid control for the hand piece 112. For example, and not by way of limitation, the fluidics subsystem 110 can manage fluid for an irrigating cannula. In general, the surgical console 100 includes a hand piece subsystem that supports one or more hand pieces 112. For example, the hand piece subsystem can manage ultrasonic oscillation for a phaco hand piece, provide laser energy to a laser hand piece, control operation of an irrigating cannula, and/or manage features of a vitrectomy hand piece.

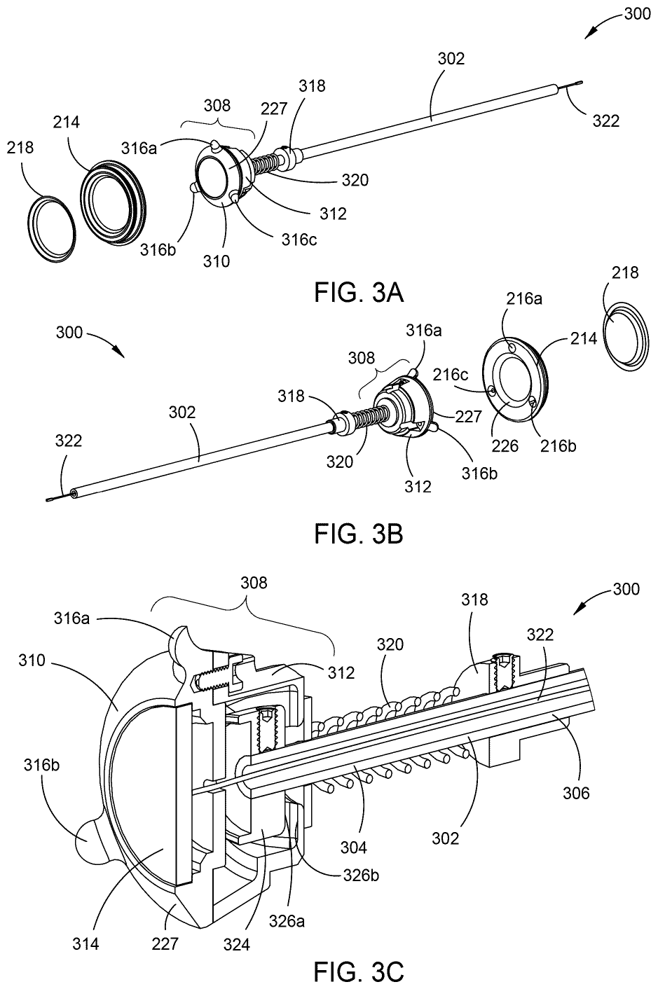

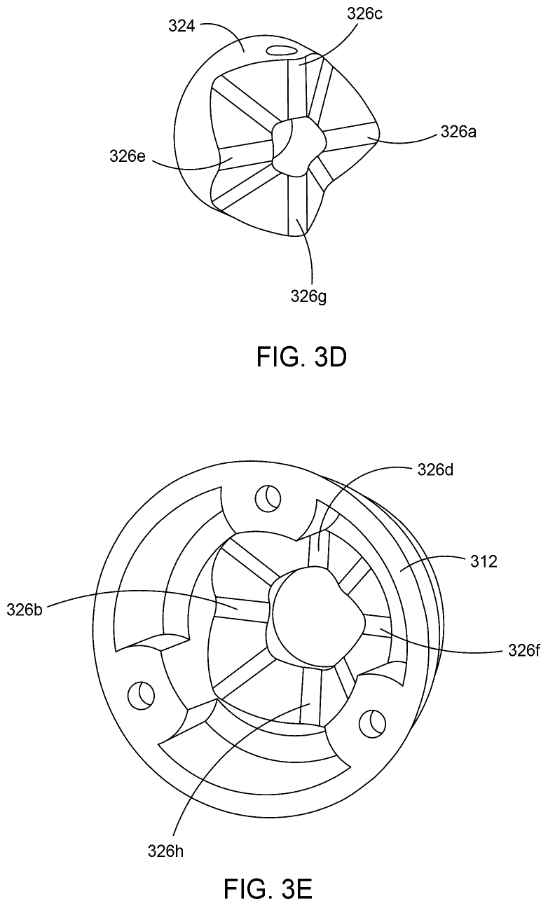

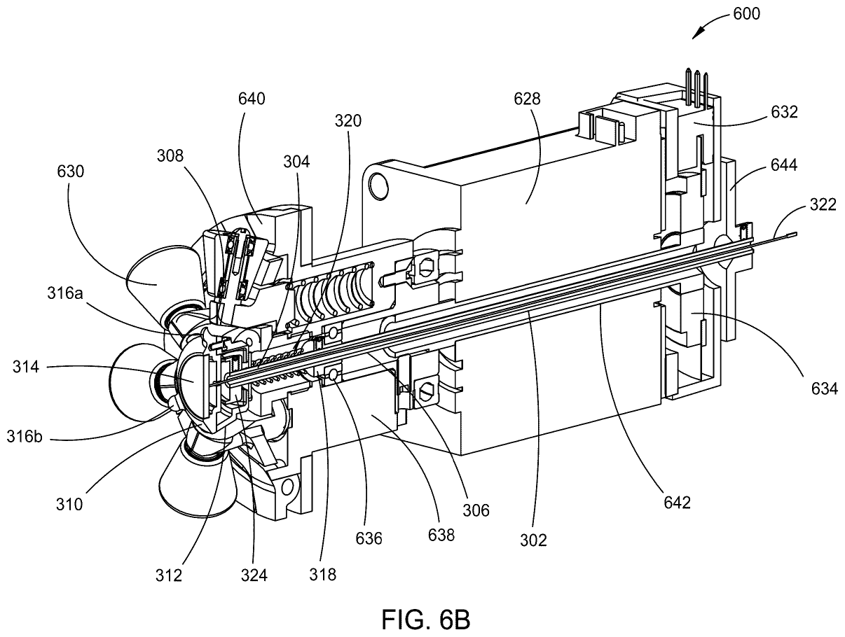

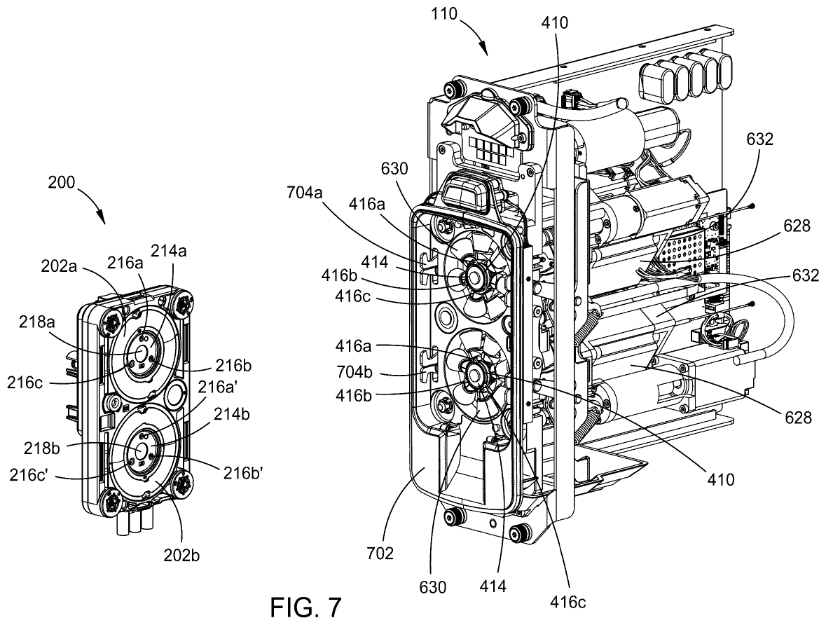

The fluidic subsystem 110 includes pump assemblies (discussed in further detail with respect to FIGS. 6-7) having sensor assemblies 108 (108a-b) therein. Sensor assemblies 108 can interact with the ophthalmic surgical system 10 via the fluidic subsystem 110. Sensor assemblies 108 are described in greater detail with respect to FIGS. 3-5. In certain embodiments, the sensor assemblies 108 can include optical sensor assemblies, inductive sensor assemblies, eddy current sensor assemblies, and the like.

Computer 103 controls operation of ophthalmic surgical system 10. Generally, computer 103 includes a processor and a memory. The memory may include any device operable to receive, store, or recall data, including, but not limited to, electronic, magnetic, or optical memory, whether volatile or non-volatile. The memory may include code stored thereon. The code may include instructions that may be executable by the processor. The code may be created, for example, using any programming language, including but not limited to, C, C++, Java, Python, Rust, or any other programming language (including assembly languages, hardware description languages, and database programming languages). In some instances, the code may be a program that, when loaded into the processor, causes the surgical console 100 to receive and process information from one or more of subsystems 106, 110, and 116, for example, providing fluid control for one or more hand pieces 112 or other devices in communication with the surgical console 100.

The processor may be, or include, a microprocessor, a microcontroller, an embedded microcontroller, a programmable digital signal processor, or any other programmable device operable to receive information from the memory or other devices in communication with the processor, computer 103, and/or console 100, and perform one or more operations on the received information. For example, the processor may send instructions to components of fluidics subsystem 110, or other devices or systems in communication with computer 103, for controlling such devices and systems. The processor may also be operable to output results based on the operations performed thereby. A display screen 104 shows data and other output results provided by the processor of computer 103. In some instances, the processor may also be or include an application specific integrated circuit, a programmable gate array, programmable array logic, or any other device of combinations of devices operable to process electric signals.

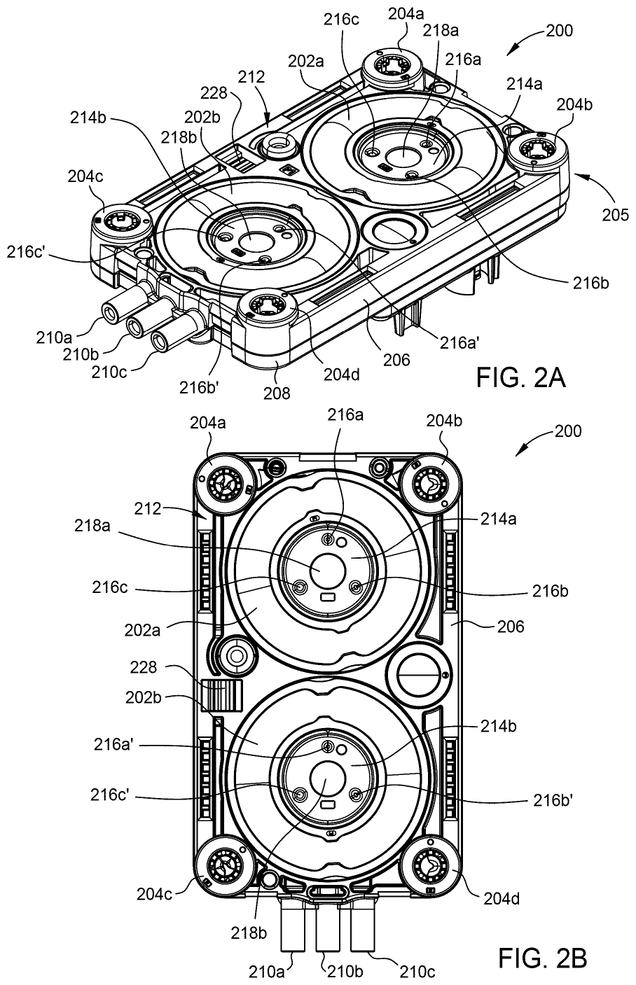

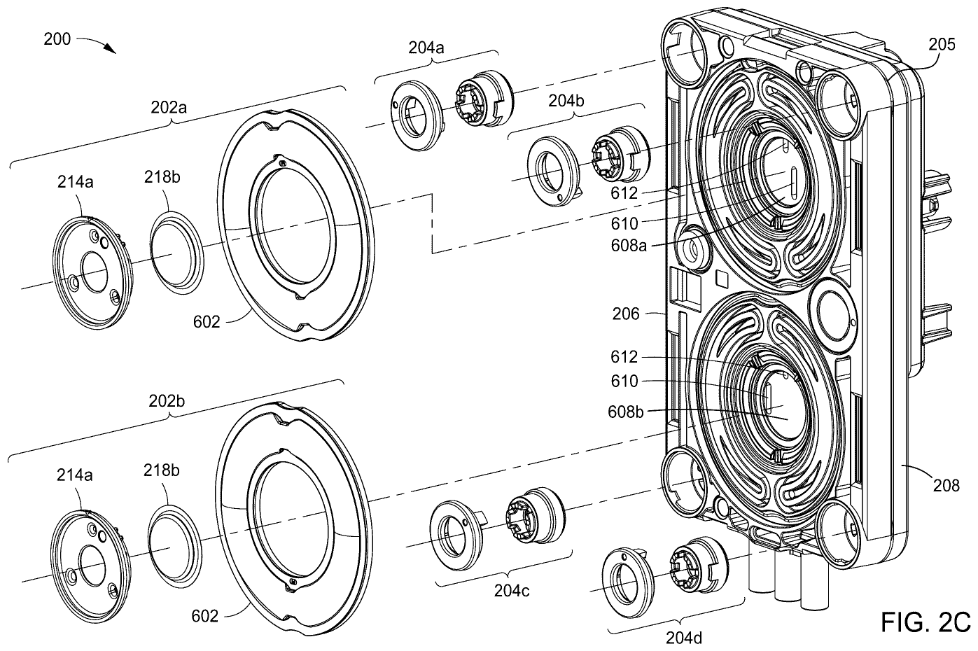

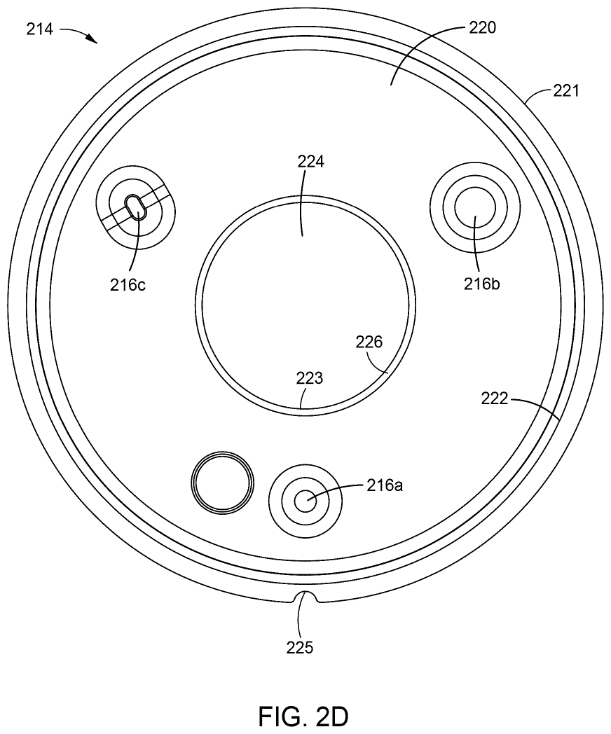

FIG. 2A illustrates a back side perspective view of a surgical cassette 200 which may be operably coupled to a console of an ophthalmic surgical system (e.g., surgical console 100 of ophthalmic surgical system 10 illustrated in FIGS. 1A-1B), according to certain embodiments. FIG. 2B illustrates a back side elevation view of surgical cassette 200 of FIG. 2A, according to certain embodiments. FIGS. 2A-2B are described together herein for clarity. Surgical cassette 200 includes two pump assemblies 202 (202a-b) which provide a source of pressure and/or vacuum for fluids being flowed within the surgical cassette 200 and four valve assemblies 204 (204a-d) which control pressure and/or fluid communication within surgical cassette 200.

In certain other embodiments, there may be only one pump assembly or more than two pump assemblies. In such embodiments, each pump assembly includes a corresponding retaining ring and diaphragm to engage with a corresponding sensor. In certain other embodiments, there may be more or less than four valve assemblies (e.g., two to six valve assemblies). In some other embodiments, an external source of pressure and/or vacuum (e.g., a venturi source) may be coupled to surgical cassette 200. In such embodiments, the external source may either be in place of, or in addition to, pump assemblies 202.

Surgical cassette 200 has a housing 205 including a base 206, a cover 208 coupled to base 206, and inlet/outlet ports 210 (210a-c) in base 206 which provide pressure and/or fluid communication between inside and outside of the housing 205. Fluid lines (e.g., tubing) may be coupled between each port 210a-c and a corresponding component of fluidics subsystem 110 and/or a corresponding hand piece 112a-c (shown in FIGS. 1A-1B). Cover 208 is disposed on a front side of the housing 205 which faces away from back side 212, and a barcode 228 is disposed within a window of back side 212.

In certain embodiments, one of a first pump assembly 202a or second pump assembly 202b provides a source of pressure (e.g., to create a driving force for fluid irrigation) while the other one of the first pump assembly 202a or second pump assembly 202b provides a source of vacuum (e.g., to create suction for fluid aspiration). The first pump assembly 202a and second pump assembly 202b may be peristaltic pumps or any other suitable type of pump for generating pressure and/or vacuum. In certain embodiments, the first pump assembly 202a and second pump assembly 202b are identical to each other.

Valve assemblies 204 are also coupled to base 206. Valve assemblies 204 function cooperatively to control pressure and/or fluid communication within and through surgical cassette 200. In the illustrated embodiments, surgical cassette 200 includes a first valve assembly 204a, a second valve assembly 204b, a third valve assembly 204c, and a fourth valve assembly 204d. As shown, in the embodiments of FIG. 2A, the four valve assemblies 204 are arranged at the four corners of base 206 and surrounding the two pump assemblies 202 which are arranged towards a center of base 206. However, in certain other embodiments, pump assemblies 202 and valve assemblies 204 may have any other suitable arrangement.

In certain embodiments, valve assemblies 204 may be operated to selectively route fluid flow between multiple internal channels of housing 205. For example, first valve assembly 204a and third valve assembly 204c may be in pressure and/or fluid communication with first pump assembly 202a and port 210a to provide aspiration (suction) through port 210a during an operation, and second valve assembly 204b and fourth valve assembly 204d may be in pressure and/or fluid communication with second pump assembly 202b and port 210c to provide irrigation (infusion) through port 210c during the same operation. In certain embodiments, port 210b may be an administration port configured to receive irrigation and/or infusion fluids to be delivered to the eye via port 210c.

Pump assemblies 202 and valve assemblies 204 are located on a back side 212 of housing 205, which is visible in FIGS. 2A-2B. Cover 208 is disposed on a front side of the housing 205 which faces away from back side 212. In certain embodiments, cover 208 may be welded, bonded, or fastened to base 206 using any suitable coupling mechanism. For example, cover 208 may be coupled to base 206 using a solid-state welding technique (e.g., ultrasonic welding in which high-frequency ultrasonic acoustic vibrations are locally applied to work parts being held together under pressure to create a solid-state weld). Back side 212 of housing 205 is configured to interface with surgical console 100 when surgical cassette 200 is coupled thereto. For example, a drive interface on a valve body of each valve assembly 204 may engage a corresponding drive mechanism of surgical console 100 for rotating the corresponding valve body. In certain embodiments, the drive mechanism is a direct drive motor which operates at lower torque with faster valve response time when compared to a geared drive motor which is conventionally used. However, the embodiments described herein may use any suitable type of drive motor.

As seen in the exploded view of FIG. 2C, each pump assembly 202a, 202b is coupled to and disposed around a first well 608a and a second well 608b, respectively, that are defined within the base 206. Each well 608a, 608b comprises an inlet 610 and an outlet 612 defined therein, the inlet 610 and outlet 612 being in fluid communication with the internal channels disposed within the surgical cassette 200. Each pump assembly 202a, 202b comprises a pump elastomer 602 which is disposed around an outer circumference of each well 608a, 608b. Disposed in a center portion of each well 608a, 608b in a substantially nested or stacked configuration is a diaphragm 218 (218a-218b) which is accommodated or disposed below a retainer ring 214 (214a-b). In certain embodiments, the retainer ring 214 is coupled to the internal surfaces of the well 608a, 608 through an ultrasonic welding process with the diaphragm 218 disposed underneath, the retainer ring 214 thereby maintaining the diaphragm 218 within each respective well 608a, 608b and providing a hermetic seal with the base 206. During use, fluid pressure within each well 608a, 608b causes deflection of the corresponding diaphragm, which can be detected and measured by a sensor of the surgical console 100 to determine fluid pressure within the internal channels and/or reservoirs of the surgical cassette 200.

Retaining rings 214 include mating elements 216 (216a-c and 216a′-c′) operable to receive a follower assembly to engage sensor components of a surgical console (e.g., surgical console 100 of ophthalmic surgical system 10 illustrated in FIGS. 1A-1B). The diaphragms 218 (218a-b) provide for sensing fluid pressure in surgical cassette 200, as described in more detail below with respect to FIGS. 3-7. In certain embodiments, the mating elements 216 may be cavities to receive a ball (e.g., ball-shaped mating elements), V-grooves, crown-shaped mating elements, cone-shaped elements, flat portions, and combinations of the same and like such that the mating elements 216 can be engaged and/or fitted with corresponding protrusions of the follower assembly. In certain embodiments, each mating element 216 can include one of a cone-shaped mating element, a V-groove, and a flat mating element. In various embodiments, each mating element 216 is spaced approximately 120° from one another. In other embodiments, the mating elements 216 can include three V-grooves aligned towards the center of the respective diaphragm 218.

In certain embodiments, surgical cassette 200 may include various internal channels, partitions, ports, surfaces, reservoirs, or fluid pockets (e.g., well 608 behind diaphragm 218 for operation of the surgical cassette 200). For example, first valve assembly 204a and third valve assembly 204c may be in pressure and/or fluid communication with first pump assembly 202a and port 210a to provide aspiration (suction) through port 210a via channels disposed within the surgical cassette 200. Additionally and/or alternatively, second valve assembly 204b and fourth valve assembly 204d may be in pressure and/or fluid communication with second pump assembly 202b and port 210c to provide irrigation (infusion) through port 210c via channels disposed within the surgical cassette 200.

In certain embodiments, diaphragm 218 may mount from the inside of the base 206 of the surgical cassette 200, and a smaller retaining ring 214 holds the diaphragm 218 in place from the inside of the base 206. In certain embodiments, a cover seals the chamber and channels, and mating features are on the base 206 of the surgical cassette 200.

FIG. 2D illustrates a top down plan view of a retainer ring that is disposed within the pump assemblies of the surgical cassette of FIGS. 2A-2B according to certain embodiments. The retainer ring 214 includes an annular or ring shaped top surface 220 defined between an outer circumference 221 and an inner circumference 223.

In certain embodiments, the retainer ring 214 includes a sloped, or conical, surface 226 disposed between the top surface 220 and the inner circumference 223. For example, in certain embodiments, a height of the top surface 220 may be higher than a height of a top edge of the inner circumference 223, and thus the surface 226 may have a negative slope towards the center of the retainer ring 214. The surface 226 may serve as a lead-in for a pressure sensor of a surgical console to facilitate proper engagement and alignment between the surgical cassette 200 and the sensor when the surgical cassette 200 is attached to the surgical console (e.g., console 100). In still other embodiments, a height of the top surface 220 may be lower than a height of a top edge of the inner circumference 223. In certain embodiments, the top surface 220 itself may be sloped, and either have a negative or positive slope towards the center of the retainer ring 214.

The top surface 220 is circumferentially surrounded by a raised lip or edge 222. Similar to the surface 226, the raised lip or edge 222 may serve as a lead-in for a pressure sensor of a surgical console. For example, the raised lip or edge 222 may serve as a lead-in for one or more protrusions or mating elements extending from the sensor.

Defined in a center of the retainer ring 214 and within the inner circumference 223 is an aperture 224, which permits the pressure sensor disposed in the surgical console 100 direct access to a surface of the diaphragm 218 disposed beneath the retainer ring 214. Also defined in the top surface 220 are a plurality of mating elements 216 (216a-c), as described above. In certain embodiments, each of the mating elements 216 include a different footprint, shape, cross-sectional depth, profile, or friction coefficient. For example, as shown in FIG. 2D, the mating element 216c includes an angled or V-slot shaped cross sectional profile, while the mating element 216b includes a substantially flat cross sectional profile, and the mating element 216a includes a conical cross sectional profile. Also seen in FIG. 2D is a notch 225 that is defined within the outer circumference 221 of the retainer ring 214. The notch 225 corresponds to a matching or correspondingly shaped featured within wells so that, when the retainer ring 214 is coupled to the surgical cassette 200, the retainer ring 214 is correctly aligned relative to the wells. In certain embodiments the notch 225 includes a substantially semi-circular or half-moon shape; however, in other embodiments, other shapes may be used.

In certain embodiments, the retainer ring 214 is formed of thermoplastic polymeric material which is resilient enough to absorb impact forces in the event of a collision with the pressure sensor or any other part of the surgical console 100, but malleable enough to bond with and provide a hermetic seal in conjunction with the diaphragm 218 when the retainer ring 214 is coupled to the base. For example, the retainer ring 214 is composed of a material which is robust enough to withstand the forces associated with ultrasonic welding and not crack or break and thus destroy any hermetic seal between it and the base. In further embodiments, the retainer ring 214 is formed of a suitable material other than a thermoplastic polymeric material.

In certain embodiments, the material of the retainer ring 214 includes or is impregnated with a “molded-in” lubricant so as to provide a desired friction coefficient across the top surface 220 and within the mating elements 216a-c. This facilitates positioning of a pressure sensor when attaching surgical cassette 200 to the surgical console 100, as the pressure sensor of the surgical console 100 may move more easily across the top surface 220 of the retainer ring 214 to its final position above the diaphragm 218. Use of “molded-in” lubricant leads to several advantages including a cleaner production process, a reduced likelihood of lubricant making contact with the end user or patient, the prevention of particulate matter from inadvertently attaching to the surface of the retainer ring 214, and ensuring that the lubricant is always in its intended location and at the appropriate amount. In certain other embodiments, a lubricant may be applied directly to the top surface 220 in addition to or instead of the retainer ring 214 itself including a lubricating material.

FIG. 3A illustrates a front side perspective view of an inductive sensor assembly 300 for sensing fluid pressure in surgical cassette 200 according to certain embodiments. FIG. 3B illustrates a back side perspective view of the inductive sensor assembly 300 for sensing fluid pressure in surgical cassette 200 according to certain embodiments. FIG. 3C illustrates an enlarged section view of the inductive sensor assembly 300 for sensing fluid pressure in surgical cassette 200 according to certain embodiments. FIGS. 3A-3C are described together herein for clarity.

Inductive sensor assembly 300 includes a tubing 302 having a first portion 304 (shown in FIG. 3C) and a second portion 306 (shown in FIG. 3C). The first portion 304 includes a follower assembly 308 attached thereon and operable to engage with retaining ring 214 of surgical cassette 200. The follower assembly 308 has a follower 310 coupled to a cup 312. In certain embodiments, the follower 310 is coupled to the cup 312 via screws, welding, press-fitting, and/or combinations of the same and like. In some embodiments, the follower 310 and the cup 312 are manufactured as a single component. As further shown in FIGS. 3A-3C, the follower assembly 308 includes a sensor component 314 positioned in a center portion of the follower 310. FIGS. 3A-3C illustrate the sensor component 314 as an inductive sensor; however, other types of sensors are readily envisioned and described in further detail herein.

Returning now to the follower assembly 308, the follower 310 includes protrusions 316 (316a-c) that engage with corresponding mating elements 216 of retaining ring 214 of surgical cassette 200 for proper alignment of the inductive sensor assembly 300. In certain embodiments, the protrusions 316 may be ball-shaped mating elements, V-groove mating elements, crown-shaped mating elements, cone-shaped mating elements, and combinations of the same and like such that protrusions 316 can be engaged and/or fitted with the corresponding mating elements 216 of retaining ring 214. In various embodiments, protrusions 316 may instead be cavities and/or recesses operable to receive a corresponding protruding portion of retaining ring 214. In certain embodiments, in addition to or alternative to the protrusions 316, the follower 310 includes a distal outer surface 227 having at least a partially conical, sloped, or ball-like shape that engages with or slides against, e.g., the surface 226 at the center of the retaining ring 214 to properly position the inductive sensor assembly 300 during attachment of the surgical cassette 200 to the surgical console 100.

The second portion 306 (shown in FIG. 3C) includes a collar 318 operable, in certain embodiments, to fit into a bearing of a pump head 640 of a fluid pump assembly within surgical console 100 operable to receive surgical cassette 200. A biasing element 320 is disposed between the follower assembly 308 and collar 318. Biasing element 320 is operable to apply a biasing force to the follower assembly 308 to facilitate engagement of the follower 310 with the surgical cassette 200 allowing the follower assembly 308 to decouple from the surgical console 100. In certain embodiments, the biasing element 320 may include a spring or an elastomer. In some embodiments, the biasing element 320 may be a spring formed from high-carbon spring steels, alloy spring steels, stainless spring steels, copper-based spring alloys, nickel-based spring alloys, and the like. As described herein, “decoupling” of the follower assembly 308 from the surgical console 100 refers to the mobility and/or movability of the follower assembly 308 relative to the surgical console 100. The decoupling of the follower assembly 308 from the surgical console 100 allows sensor components within the follower assembly 308 to follow the diaphragm 218 of the surgical cassette 200, as described in further detail below. In certain other embodiments, the second portion 306 or the collar 318 may be fit directly to the fluidics subsystem 110 or another structural element, such as face plate 118 to provide alternate sensor placement options that are not within a pump center.

As shown in FIG. 3C, follower assembly 308 includes a mechanical stop 324 positioned between the follower 310 and the cup 312. The mechanical stop 324 is disposed at an end of the first portion 304 of tubing 302 and includes first alignment mechanism 326a (shown in FIGS. 3D-3E) that correspond with second alignment mechanism 326b (shown in FIGS. 3D-3E) in an inside portion of cup 312. First alignment mechanism 326a and second, corresponding alignment mechanism 326b, are collectively referred to herein as alignment mechanism 326. In certain embodiments, the mechanical stop 324 is positioned between the follower 310 and the cup 312 as the biasing element 320 compresses and decompresses, thereby biasing the follower assembly 308 forward. In certain embodiments, the alignment mechanism 326 may include V-grooves operable to receive one another (i.e., receive first alignment mechanism 326a into second alignment mechanism 326b, or vice versa). In some embodiments, the alignment mechanism 326 may include any type of mechanism to maintain proper alignment between the mechanical stop 324 and the cup 312. Tubing 302 includes a cable 322 disposed therein to connect the sensor component 314 to electronic circuitry to operate the sensor component 314. The tubing 302 of the inductive sensor assembly 300 protects cable 322 as the tubing 302 extends through a pump head 640 and a hollow shaft of a pump motor and encoder, described in further detail below with respect to FIGS. 6-7. In certain other embodiments, the tubing 302 length is considerably less, and the tubing is coupled within the front opening 702 of the fluidics subsystem 110 to allow engagement with the surgical cassette 200.

During clamping of the surgical cassette 200 to the surgical console 100, the surgical cassette 200 is pulled into the follower assembly 308 and the retaining ring 214 of the surgical cassette 200 contacts the follower 310, causing the biasing element 320 to compress and move the follower assembly 308 towards the surgical console 100. The engagement of the sensor component 314 to the surgical cassette 200, and the decoupling of the sensor component 314 from the surgical console 100, as provided by the biasing element 320, allows the sensor component 314 to follow the diaphragm 218 of the surgical cassette 200 while the surgical cassette 200 shifts, vibrates, expands, or contracts during a surgical procedure. In other words, when the surgical cassette 200 is clamped to the surgical console 100, the decoupling of the sensor component 314 enables the sensor component 314 to be positioned at a constant distance from a base of the diaphragm 218 of the surgical cassette 200. Since the diaphragm 218 deflects as a result of pressure changes within well 608 behind the diaphragm 218, measuring a change in distance between the sensor component 314 and the diaphragm 218 enables consistent precise and accurate measurements of fluidic pressure within the surgical cassette 200. When the surgical cassette 200 is removed, the sensor component 314 is configured to bottom out and align to a position to be ready for another surgical cassette 200. This is accomplished using mechanical stop 324 that is disposed within the follower assembly 308.

In certain embodiments, the inductive sensor assembly 300 is positioned in the center of a fluid pump assembly, described in further detail below with respect to FIGS. 6-7, and the diaphragm 218 is positioned in the center of the pump assembly 202 of the surgical cassette 200 to minimize the size of both the surgical cassette 200 and fluidics subsystem 110 of the surgical console 100. The inductive sensor assembly 300 is held concentric to the pump head 640 via a bearing 636 to allow the pump head 640 to rotate while keeping the sensor assembly relatively stationary, described in further detail below with respect to FIGS. 6-7.

FIG. 3D illustrates a backside perspective view of mechanical stop 324. FIG. 3E illustrates a front side perspective view of cup 312. FIGS. 3D-3E are described together herein for clarity. Mechanical stop 324 includes first alignment mechanism 326a, third alignment mechanism 326c, fifth alignment mechanism 326e, and seventh alignment mechanism 326g that correspond to second, corresponding alignment mechanism 326b, fourth, corresponding alignment mechanism 326d, sixth, corresponding alignment mechanism 326f, and eighth alignment mechanism 326h of an inside portion of cup 312 (collectively referred to herein as alignment mechanism 326). In certain embodiments, the alignment mechanism 326 may include V-grooves operable to receive one another (i.e., receive first alignment mechanism 326a into second, corresponding alignment mechanism 326b, or vice versa). In some embodiments, the alignment mechanism 326 may include any type of mechanism to maintain proper alignment between the mechanical stop 324 and the cup 312.

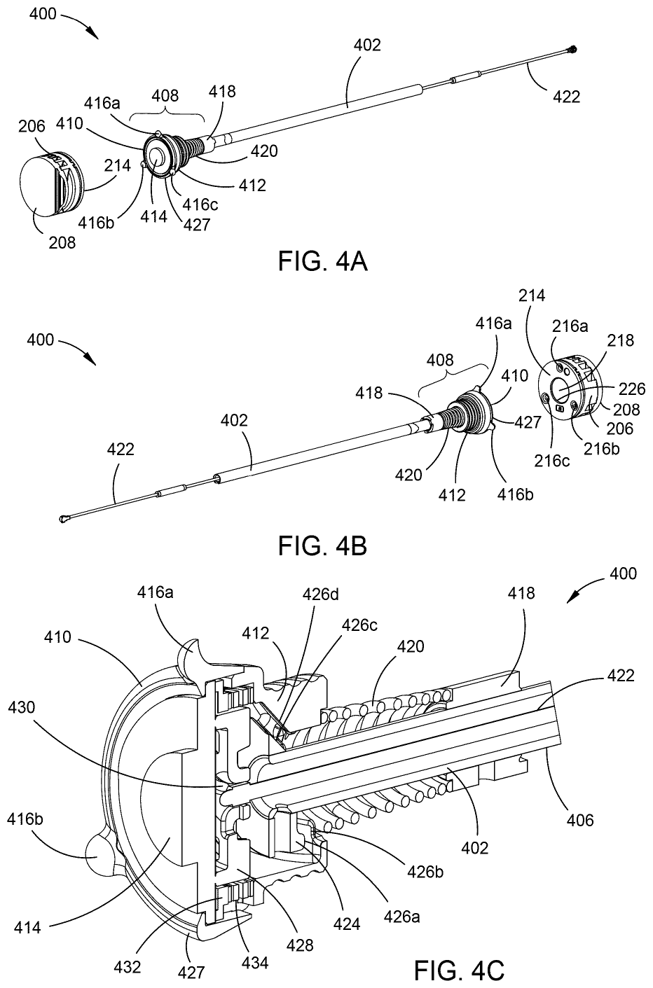

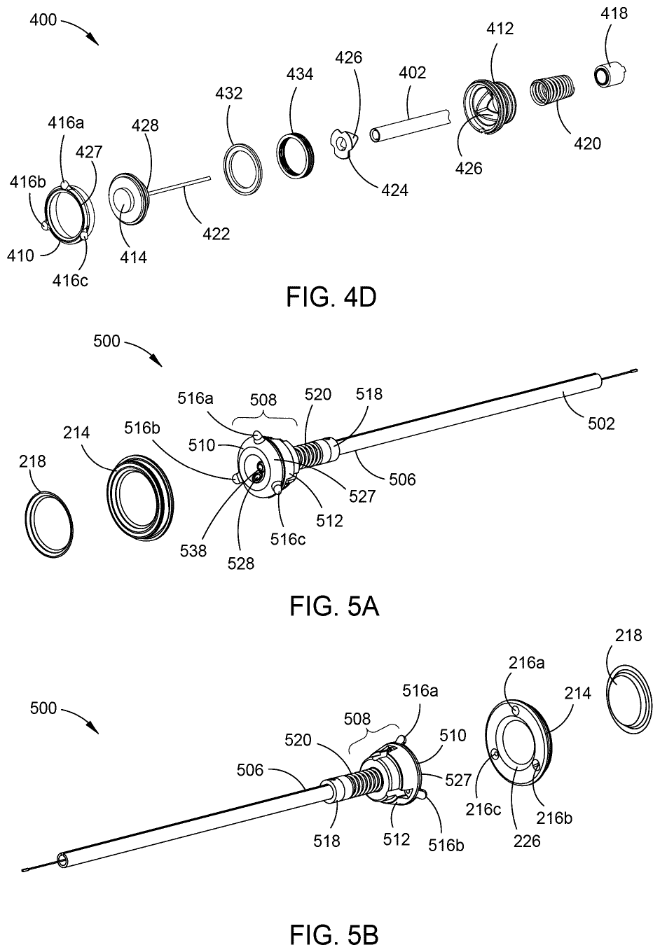

FIG. 4A illustrates a front side perspective view of an eddy current sensor assembly 400 for sensing fluid pressure in surgical cassette 200 according to certain embodiments. FIG. 4B illustrates a back side perspective view of the eddy current sensor assembly 400 for sensing fluid pressure in surgical cassette 200 according to certain embodiments. FIG. 4C illustrates an enlarged view of the eddy current sensor assembly 400 for sensing fluid pressure in surgical cassette 200 according to certain embodiments. FIG. 4D illustrates an exploded view of the eddy current sensor assembly 400 for sensing fluid pressure in surgical cassette 200 according to certain embodiments. FIGS. 4A-4D are described together herein for clarity.

Eddy current sensor assembly 400 includes a tubing 402 having a first portion 404 (shown in FIG. 4C) and a second portion 406 (shown in FIG. 4C). The first portion 404 (shown in FIG. 4C) includes a follower assembly 408 attached thereon operable to engage with retaining ring 214 of surgical cassette 200. For illustrative purpose, base 206 and cover 208 of the surgical cassette 200 are shown with retaining ring 214 in FIGS. 4A-4B. The follower assembly 408 has a follower 410 coupled to a cup 412. In certain embodiments, the follower 410 is coupled to the cup 412 via screws, welding, press-fitting, and/or combinations of the same and like. In some embodiments, the follower 410 and the cup 412 are manufactured as a single component. As further shown in FIGS. 4A-4D, the follower assembly 408 includes a sensor component 414 positioned in a center portion of the follower 410. The sensor component 414 includes a printed circuit board assembly (PCBA) with a coil activatable by high frequency alternating current and signal conditioning electronics capable of sensing inductance, impedance variation, or a resonant frequency change in the coil as a gap between the diaphragm 218 and the position coil changes. Translation of that variation into a displacement signal is then correlated to fluid pressure. FIGS. 4A-4D illustrate the sensor component 414 as an eddy current sensor; however, other sensors are readily envisioned and described in further detail herein.

Returning now to the follower assembly 408, the follower 410 includes protrusions 416 (416a-c) that engage with corresponding mating elements 216 of retaining ring 214. In certain embodiments, the protrusions 416 may be ball-shaped mating elements, V-groove mating elements, crown-shaped mating elements, cone-shaped mating elements, and combinations of the same and like such that protrusions 416 can be engaged and/or fitted with the corresponding mating elements 216 of retaining ring 214. In various embodiments, protrusions 316 may instead be cavities and/or recesses operable to receive a corresponding protruding portion of retaining ring 214. In certain embodiments, in addition to or alternative to the protrusions 416, the follower 410 includes a distal outer surface 427 having at least a partially conical, sloped, or ball-like shape that engages with or slides against, e.g., the surface 226 at the center of the retaining ring 214 to properly position the eddy current sensor assembly 400 during attachment of the surgical cassette 200 to the surgical console 100.

The second portion 406 (shown in FIG. 4C) includes a collar 418, in certain embodiments, operable to fit into a bearing 636 of a pump head 640 of a fluid pump assembly within surgical console 100 operable to receive surgical cassette 200. A biasing element 420 is disposed between the follower assembly 408 and collar 418. Biasing element 420 is operable to apply a biasing force to the follower assembly 408 to facilitate engagement of the follower 410 with the surgical cassette 200 allowing the follower assembly 408 to decouple from the surgical console 100. In certain embodiments, the biasing element 420 may include a spring or an elastomer. In some embodiments, the biasing element 420 may be a spring formed from high-carbon spring steels, alloy spring steels, stainless spring steels, copper-based spring alloys, nickel-based spring alloys, and the like.

As shown in FIGS. 4C-4D, follower assembly 408 includes a mechanical stop 424 positioned between the follower 410 and the cup 412. The mechanical stop 424 is disposed at an end of the first portion 404 of tubing 402, and includes first alignment mechanism 426a (similar to alignment mechanism 326 as described in FIGS. 3D-3E) that correspond with second alignment mechanism 426b (similar to alignment mechanism 326 as described in FIGS. 3D-3E) in an inside portion of cup 412, and third alignment mechanism 426c that correspond with fourth alignment mechanism 426d in the inside portion of cup 412. First alignment mechanism 426a, corresponding second alignment mechanism 426b, third alignment mechanism 426c, and corresponding fourth alignment mechanism 426d are collectively referred to herein as alignment mechanism 426. In certain embodiments, the mechanical stop 424 is positioned between the follower 410 and the cup 412 as the biasing element 420 compresses and decompresses thereby biasing the follower assembly 408 forward. In certain embodiments, the alignment mechanism 426 may include V-grooves operable to receive one another (i.e., receive first alignment mechanism 426a into second alignment mechanism 426b and third alignment mechanism 426c into fourth alignment mechanism 426d, or vice versa). In some embodiments, the alignment mechanism 426 may include any type of mechanism to maintain proper alignment between the mechanical stop 424 and the cup 412.

Tubing 402 includes a cable 422 disposed therein to connect the sensor component 414 to electronic circuitry to operate the sensor component 414. The tubing 402 of the eddy current sensor assembly 400 protects cable 422 as the tubing 402 extends through a pump head 640 and a hollow shaft of a pump motor and encoder, described in further detail below with respect to FIGS. 6-7. A strain relief 428 having a radial opening 430 to interface with the cable 422 is disposed between the mechanical stop 424 and the sensor component 414. The strain relief 428 is bonded to the sensor component 414 and holds at least a portion of the cable 422 to reduce tension of the cable 422 between the strain relief 428 and the sensor component 414. As such, the strain relief 428 reduces strain on cable 422 at the mounting point to the sensor component 414 during movement of the follower assembly 408, such as, for example, during a surgical operation, while clamping the surgical cassette 200 to the surgical console 100, and/or unclamping the surgical cassette 200 from the surgical console 100. PCBA support 432 and PCBA spring 434 are positioned about an outer radius of the strain relief 428 and provide clearance thereof.

During clamping of the surgical cassette 200 to the surgical console 100, the surgical cassette 200 is pulled into the follower assembly 408 and the retaining ring 214 of the surgical cassette 200 contacts the follower 410, causing the biasing element 420 to compress and move the follower assembly 408 towards the surgical console 100. The engagement of the sensor component 414 to the surgical cassette 200, and the decoupling of the sensor component 414 from the surgical console 100, as provided by the biasing element 420, allows the sensor component 414 to follow the diaphragm 218 of the surgical cassette 200 while the surgical cassette 200 shifts, vibrates, expands, or contracts during a surgical procedure. In other words, when the surgical cassette 200 is clamped to the surgical console 100, the decoupling of the sensor component 414 enables the sensor component 414 to be positioned at a constant distance from a base of the diaphragm 218 of the surgical cassette 200. Since the diaphragm 218 deflects as pressure changes within well 608 behind the diaphragm 218, measuring a change in distance between the sensor component 414 and the diaphragm 218 enables consistent, precise, and accurate measurements of fluidic pressure within the surgical cassette 200. When the surgical cassette 200 is removed, the sensor component 414 is configured to bottom out and align to a position to be ready for another surgical cassette 200. This is accomplished using mechanical stop 424 that is disposed within the follower assembly 408.

In certain embodiments, the eddy current sensor assembly 400 is positioned in the center of a fluid pump assembly, described in further detail below with respect to FIGS. 6-7, and the diaphragm 218 is positioned in the center of the pump assembly 202 of the surgical cassette 200 to minimize the size of both the surgical cassette 200 and fluidics subsystem 110 of the surgical console 100. The collar 418 of the eddy current sensor assembly 400 is held concentric to the pump head 640 via a bearing 636 and allows the pump head 640 to rotate while keeping the collar 418, tube 402, and mechanical stop 424 of the eddy current sensor assembly 400 relatively stationary, described in further detail below with respect to FIGS. 6-7.

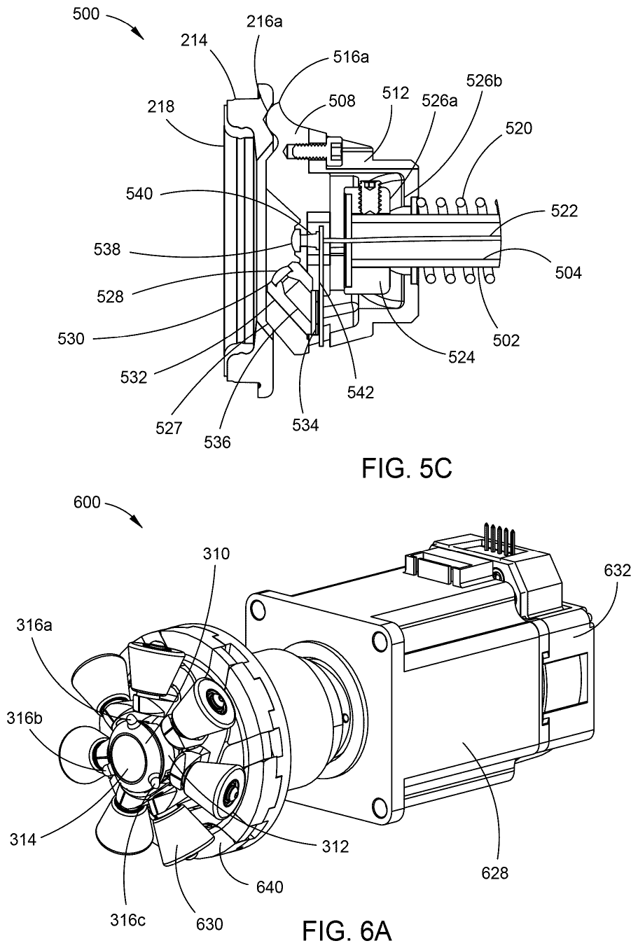

FIG. 5A illustrates a front side perspective view of an optical sensor assembly 500 for sensing fluid pressure in surgical cassette 200 according to certain embodiments. FIG. 5B illustrates a back side perspective view of the optical sensor assembly 500 for sensing fluid pressure in surgical cassette 200 according to certain embodiments. FIG. 5C illustrates an enlarged view of the optical sensor assembly 500 for sensing fluid pressure in surgical cassette 200 according to certain embodiments. FIGS. 5A-5C are described together herein for clarity.

Optical sensor assembly 500 includes a tubing 502 having a first portion 504 (shown in FIG. 5C) and a second portion 506 (shown in FIG. 5B). The first portion 504 (shown in FIG. 5C) includes a follower assembly 508 attached thereon operable to engage with retaining ring 214 of surgical cassette 200.

The follower assembly 508 has a follower 510 coupled to a cup 512. In certain embodiments, the follower 510 is coupled to the cup 512 via screws, welding, press-fitting, and/or combinations of the same and like. In some embodiments, the follower 510 and the cup 512 are manufactured as a single component. As further shown in FIGS. 3A-3C, the follower 510 includes protrusions 516 (516a-c) that engage with corresponding mating elements 216 of retaining ring 214 of surgical cassette 200. In certain embodiments, the protrusions 516 may be ball-shaped mating elements, V-groove mating elements, crown-shaped mating elements, cone-shaped mating elements, and combinations of the same and like such that protrusions 516 can be engaged and/or fitted with the corresponding mating elements 216 of retaining ring 214. In various other embodiments, protrusions 316 may instead be cavities and/or recesses operable to receive a corresponding protruding portion of retaining ring 214. In certain embodiments, in addition to or alternative to the protrusions 516, the follower 510 includes a distal outer surface 527 having at least a partially conical, sloped, or ball-like shape that engages with or slides against, e.g., the surface 226 at the center of the retaining ring 214 to properly position the optical sensor assembly 500 during attachment of the surgical cassette 200 to the surgical console 100.

The second portion 506 (shown in FIG. 5B) includes a collar 518 operable, in certain embodiments, to fit into a bearing of a pump head 640 of a fluid pump assembly within surgical console 100 operable to receive surgical cassette 200. A biasing element 520 is disposed between the follower assembly 508 and collar 518. Biasing element 520 is operable to apply a biasing force to the follower assembly 508 to facilitate engagement of the follower 510 with the surgical cassette 200 allowing the follower assembly 508 to decouple from the surgical console 100. In certain embodiments, the biasing element 520 may include a spring or an elastomer. In some embodiments, the biasing element 520 may be a spring formed from high-carbon spring steels, alloy spring steels, stainless spring steels, copper-based spring alloys, nickel-based spring alloys, and the like.

As shown in FIG. 5C, follower assembly 508 includes a mechanical stop 524 positioned between the follower 510 and the cup 512. The mechanical stop 524 is disposed at an end of the first portion 504 of tubing 502 and includes first alignment mechanism 526a (similar to alignment mechanism 326 as described in FIGS. 3D-3E) that correspond with second alignment mechanism 526b (similar to alignment mechanism 326 as described in FIGS. 3D-3E) in an inside portion of cup 512. First alignment mechanism 526a and second corresponding alignment mechanism 526b are collectively referred to herein as alignment mechanism 526. In certain embodiments, the mechanical stop 524 moves between the follower 510 and the cup 512 as the biasing element 520 compresses and decompresses thereby moving the follower assembly 508. In certain embodiments, the alignment mechanism 526 may include V-grooves operable to receive one another (i.e., receive first alignment mechanism 526a into second alignment mechanism 526b, or vice versa). In some embodiments, the alignment mechanism 526 may include any type of mechanism to maintain proper alignment between the mechanical stop 524 and the cup 512.

Sensor components of the optical sensor assembly 500 include a camera lens 528 disposed on a first end 530 of a lens tube 532 within the follower 510, and a camera sensor 534 disposed on a second end 536 of the lens tube 532. Camera lens 528 is configured to receive and propagate images from the diaphragm 218 through the lens tube 532 such that the camera sensor 534 receives the images from the diaphragm 218. Follower 510 includes an illumination lens 538 disposed in a center portion of the follower 510, and an illumination source 540 disposed behind the illumination lens 538. In certain embodiments, the illumination source 540 can include, for example, one or more light emitting diodes (LEDs). Illumination source 540 illuminates the diaphragm 218 through the illumination lens 538 such that the camera sensor 534 can detect changes in the diaphragm 218 through the camera lens 528. PCBA 542 is operably coupled to the camera sensor 534 and the illumination source 540. In certain embodiments, PCBA 542 provides electronic communication to and/or from the camera sensor 534 and the illumination source 540 via cable 522. FIGS. 5A-5C illustrate the sensor components as an optical sensor; however, other sensors are readily envisioned and described in further detail herein. Tubing 502 includes the cable 522 disposed therein to connect the PCBA 542 to electronic circuitry to operate the camera sensor 534 and the illumination source 540. The tubing 502 of the optical sensor assembly 500 protects cable 522 as the tubing 502 extends through a pump head 640 and a hollow shaft of a pump motor and encoder, described in further detail below with respect to FIGS. 6-7.

During clamping of the surgical cassette 200 to the surgical console 100, the surgical cassette 200 is pulled into the follower assembly 508 and the retaining ring 214 of the surgical cassette 200 contacts portions of the follower 310, causing the biasing element 520 to compress and move the follower assembly 508 towards the surgical console 100. The engagement of the portions of the sensor components of the optical sensor assembly 500 to the surgical cassette 200, and the decoupling of the portions of the sensor components of the optical sensor assembly 500 from the surgical console 100, as provided by the biasing element 520, allows the portions of the sensor components of the optical sensor assembly 500 to follow the diaphragm 218 of the surgical cassette 200 while the surgical cassette 200 shifts, vibrates, expands, or contracts during a surgical procedure. In other words, when the surgical cassette 200 is clamped to the surgical console 100, the decoupling of the sensor components of the optical sensor assembly 500 enables the sensor components of the optical sensor assembly 500 to be positioned at a constant distance from a base of the diaphragm 218 of the surgical cassette 200. Since the diaphragm 218 deflects as pressure changes within surgical cassette 200, measuring a change in distance between the optical sensor assembly 500 and the diaphragm 218 enables consistent, precise, and accurate measurements of fluidic pressure within the surgical cassette 200. When the surgical cassette 200 is removed, the portions of the sensor components of the optical sensor assembly 500 are configured to bottom out and align to a position to be ready for another surgical cassette 200. This is accomplished using mechanical stop 524 that is disposed within the follower assembly 508.

In certain embodiments, the optical sensor assembly 500 is positioned in the center of a fluid pump assembly, described in further detail below with respect to FIGS. 6-7, and the diaphragm 218 is positioned in the center of the pump assembly 202 of the surgical cassette 200 to minimize the size of both the surgical cassette 200 and fluidics subsystem 110 of the surgical console 100. The collar 518 of the optical sensor assembly 500 is held concentric to the pump head 640 via a bearing 636 to allow the pump head to rotate while keeping the collar 518, tube 502, and mechanical stop 524 of the sensor assembly relatively stationary, described in further detail below with respect to FIGS. 6-7.

FIG. 6A illustrates a front side perspective view of a fluid pump assembly 600 having the inductive sensor assembly 300 therein for sensing fluid pressure in surgical cassette 200 according to certain embodiments. FIG. 6B illustrates an enlarged view of the fluid pump assembly 600 according to certain embodiments. FIGS. 6A-6B, with reference to FIGS. 3A-3C of inductive sensor assembly 300, are described together herein for clarity. Although illustrated and described with reference to the inductive sensor assembly 300, the arrangement/configuration of the fluid pump assembly 600 in FIGS. 6A-6B can also be utilized with the eddy current sensor assembly 400 and the optical sensor assembly 500 described herein.

Fluid pump assembly 600 includes a motor 628 having rollers 630 disposed on a front side of the fluid pump assembly 600 and an encoder 632 disposed on a back side of the fluid pump assembly 600. Motor 628 drives the pump head 640 such that rollers 630 contact and roll, in a circular motion, against the annular pump elastomer of pump assembly 202 of the surgical cassette 200, which provides a source of pressure and/or vacuum for controlling pressure and/or fluid communication within surgical cassette 200. Encoder 632 provides information to a computer of a surgical console, e.g., computer 103 of surgical console 100, about the shaft speed and/or position of the motor 628 utilizing an encoder disk 634 disposed therein. The fluid pump assembly 600 includes an inductive sensor assembly 300, as shown in FIGS. 3A-3C, disposed therein. In certain embodiments, the fluid pump assembly 600 may include, without limitation, a pump head with a hollow body, a hollow center screw, and/or a center support bearing.

Inductive sensor assembly includes tubing 302 having the first portion 304 and the second portion 306 disposed through a center portion of the motor 628 and encoder 632. The first portion 304 includes the follower assembly 308 disposed within a pump head 640 operable to engage with retaining ring 214 of surgical cassette 200. The follower assembly 308 has the follower 310 coupled to the cup 312. Follower assembly 308 includes the sensor component 314. The follower 310 includes protrusions 316 (316a-c) that engage with corresponding mating elements 216 of retaining ring 214. The second portion 306 includes the collar 318 operable to fit into a bearing 636 of a pump head 640 of the fluid pump assembly 600 within surgical console 100 operable to receive surgical cassette 200. In certain embodiments, the bearing 636 may be a hollow support bearing. The biasing element 320 is disposed between the follower assembly 308 and collar 318. Biasing element 320 is operable to apply a biasing force to the follower assembly 308 to engage the follower 310 with the surgical cassette 200.

Follower assembly 308 includes the mechanical stop 324 positioned between the follower 310 and the cup 312. The mechanical stop 324 is disposed at an end of the first portion 304 of tubing 302. Tubing 302 includes the cable 322 disposed therein to connect the sensor component 314 to electronic circuitry to operate the sensor component 314 as shown in FIG. 1B.

In certain embodiments, the inductive sensor assembly 300 is positioned in the center of fluid pump assembly 600. The collar 318 of the inductive sensor assembly 300 is held concentric to the pump head 640 via bearing 636 to allow the pump head 640 to rotate while keeping the collar 318 of the inductive sensor assembly 300 relatively stationary. The tubing 302 of the inductive sensor assembly 300 protects cable 322 as the tubing 302 extends through the pump head 640 and a hollow shaft 642 of the motor 628 and encoder 632. A sensor support bracket 644 mounts to a back end of the motor 628 and extends behind the encoder 632 where the sensor support bracket 644 secures a far end of the tubing 302. Cable 322 extends out the tubing 302 and connects to the sensor components mounted to the surgical console 100.

In certain embodiments, the fluid pump assembly may be a pump unit assembly for driving fluid in a surgical cassette with a provision for ancillary assemblies at the pump center. In such embodiments, the pump unit assembly may include, for example, a hollow body pump head with center support bearing, a hollow shaft motor, a hollow disc encoder, and a rear support bracket extending from the rear of the motor to the rear of the encoder with a center hole aligned with the motor shaft and configured to support the far end of an ancillary component.

FIG. 7 illustrates a front side perspective view of two fluid pump assemblies 600, having two eddy current sensor assemblies 400, installed in the fluidic subsystem 110 of the surgical console 100 and an associated surgical cassette 200 according to certain embodiments. For clarity, FIG. 7 is described with reference to FIGS. 2A-2B, FIGS. 4A-4D, and FIGS. 6A-6B of surgical cassette 200, eddy current sensor assembly 400, and fluid pump assembly 600, respectively. Each fluid pump assembly 600 includes an eddy current sensor assembly 400 disposed therein. Although illustrated and described with reference to the eddy current sensor assembly 400, the arrangement/configuration of the fluid pump assembly 600 in FIGS. 6A-3C can also be utilized with the inductive sensor assembly 300 and the optical sensor assembly 500 described herein.

The associated surgical cassette 200 is operably received in a front opening 702 of the fluidic subsystem 110 via clamping elements 704 (704a and 704b). When the surgical cassette 200 is received in the front opening 702, mating elements 216 (216a-c and 216a′-c′) of retaining rings 214 (214a-b) mate with protrusions 416 (416a-416c) of follower 410 to facilitate alignment of the follower 410 of the follower assembly 408 with the surgical cassette 200, and thus, proper engagement of the surgical cassette 200 with the fluidic subsystem 110. As the surgical cassette 200 is clamped into the surgical console via clamping elements 704, the surgical cassette 200 is pulled into the follower assembly 408 and the retaining ring 214 of the surgical cassette 200 contacts the follower 410, causing biasing element 420 to compress and move the follower assembly 408 towards the surgical console 100. The engagement of the sensor component 414 to the surgical cassette 200, and the decoupling of the sensor component 414 from the surgical console 100, provided by the biasing element 420, allows the sensor components 414 to follow the retaining ring 214 and diaphragm 218 of the surgical cassette 200 while the surgical cassette shifts, vibrates, expands, or contracts during a surgical procedure. In other words, when the surgical cassette 200 is clamped to the surgical console 100, the decoupling of the sensor component 414 enables the sensor component 414 to be positioned at a constant distance from the retaining ring 214 and diaphragm 218 of the surgical cassette 200. Since the diaphragm 218 deflects as pressure changes within the surgical cassette 200, measuring a change in distance between the sensor component 414 and the diaphragm 218 enables consistent, precise, and accurate measurements of fluidic pressure within the surgical cassette 200. When the surgical cassette 200 is removed, the sensor component 414 is configured to move back out and align to a position to be ready for another surgical cassette 200. This is accomplished using mechanical stop 424 that is attached to the fixed tube 402 within the follower assembly 408.

In certain embodiments, the computer 103 of the surgical console 100 may be configured to utilize calibration data of the surgical cassette 200 to convert detected and measured deflection of the diaphragm 218 to fluidic pressure within the surgical cassette 200. In certain embodiments, calibration data for the surgical cassette 200 may be attributed with, or linked to, the barcode 228 of surgical cassette 200. In various embodiments, the calibration data for the surgical cassette 200 is generated and then captured and/or stored on computer 103, and/or on a network server or other storage device in communication with the computer 103, at the time of manufacture. In certain embodiments, the calibration data is specific to each surgical cassette 200. In some embodiments, the barcode 228 may include, for example, a one-, two-or three-dimensional barcode, a data matrix barcode, or a Quick Response code. In certain embodiments, calibration data for the surgical cassette 200 is embedded onto the surgical cassette 200 in the barcode 228.

In certain embodiments, upon attachment of the surgical cassette 200 to the surgical console 100, the barcode reader 120 “reads” the barcode 228 (e.g., captures images of the barcode 228) before performance of surgical operations with the surgical console 100. In certain embodiments, when an image of the barcode 228 is captured, the computer 103 retrieves the calibration data associated with the image of the barcode 228, and thereafter correlates measured deflection of diaphragm 218, caused by pressure changes within the surgical cassette 200, into fluidic pressure based, at least in part, on the calibration data linked to barcode 228.

As described above, once the surgical cassette 200 is engaged with the fluidic subsystem 110, sensor component 414 interacts with diaphragm 218 for sensing fluid pressure in the surgical cassette 200. Rollers 630 contact pump assembly 202 (202a-b) which provide a source of pressure and/or vacuum which control pressure and/or fluid communication within surgical cassette 200.

In certain embodiments, the eddy current sensor assembly 400 is positioned in the center of fluid pump assembly 600 and the diaphragm 218 is positioned in the center of the pump assembly 202 of the surgical cassette 200 to minimize the size of both the surgical cassette 200 and a fluidics subsystem 110 of the surgical console 100. The collar 418 of the eddy current sensor assembly 400 is held concentric to the pump head 640 via a bearing 636 to allow the pump head 640 to rotate while keeping the collar 418 of the eddy current sensor assembly 400 relatively stationary. Tubing 402 of the eddy current sensor assembly 400 protects cable 422 as the tubing 402 extends through the pump head 640 and the hollow shaft 642 of the motor 628 and encoder 632. Sensor support bracket 644 mounts to the back end of the motor 628 and extends behind the encoder 632 where the sensor support bracket 644 secures the far end of the tubing 402. The cable 422 extends out the tubing 402 and connects to the sensor control components mounted to the surgical console 100.

As illustrated above, during clamping of the surgical cassette to the surgical console, the surgical cassette is pulled into the follower assembly so that the retaining ring of the surgical cassette contacts the follower, causing the biasing element to compress and move the follower assembly towards the surgical console. The engagement of the sensor component to the surgical cassette, and the decoupling of the sensor component from the surgical console, as provided by the biasing element, allows the sensor component to follow the retaining ring and diaphragm of the surgical cassette while the surgical cassette shifts, vibrates, expands, or contracts during a surgical procedure.

As such, when the surgical cassette is clamped to the surgical console, the decoupling of the sensor component enables the sensor component to be positioned at a constant distance from a base of the retaining ring and the diaphragm of the surgical cassette. Since the diaphragm deflects as pressure changes, measuring a change in distance between the sensor component and the diaphragm 218 enables consistent, precise, and accurate measurements of fluidic pressure within the surgical cassette. When the surgical cassette is removed, the sensor component is configured to move back out and align to a position to be ready for another surgical cassette. This is accomplished using mechanical stop that is disposed within the follower assembly, thus allowing for quick changing of surgical cassettes within the surgical console.

In brief, the embodiments described herein provide improved stability of fluidic measurements by mechanically decoupling the sensor component from the surgical console while mechanically coupling the sensor component to the surgical cassette at the location of the diaphragm. This reduces unwanted relative motion between the diaphragm and sensor components caused from various phenomena in typical designs, such as, for example, vibration, thermal expansion, component flexing, alignment limitations, and mechanical disturbances.

Although various embodiments of the present disclosure have been illustrated in the accompanying Drawings and described in the foregoing Detailed Description, it will be understood that the present disclosure is not limited to the embodiments disclosed herein, but is capable of numerous rearrangements, modifications, and substitutions without departing from the spirit of the disclosure as set forth herein.

The term “substantially” is defined as largely but not necessarily wholly what is specified, as understood by a person of ordinary skill in the art. In any disclosed embodiment, the terms “substantially”, “approximately”, “generally”, and “about” may be substituted with “within [a percentage] of” what is specified, where the percentage includes 0.1, 1, 5, and 10 percent.

The foregoing outlines features of several embodiments so that those skilled in the art may better understand the aspects of the disclosure. Those skilled in the art should appreciate that they may readily use the disclosure as a basis for designing or modifying other processes and structures for carrying out the same purposes and/or achieving the same advantages of the embodiments introduced herein. Those skilled in the art should also realize that such equivalent constructions do not depart from the spirit and scope of the disclosure, and that they may make various changes, substitutions, and alterations herein without departing from the spirit and scope of the disclosure. The scope of the invention should be determined only by the language of the claims that follow. The term “comprising” within the claims is intended to mean “including at least” such that the recited listing of elements in a claim are an open group. The terms “a”, “an”, and other singular terms are intended to include the plural forms thereof unless specifically excluded. Conditional language used herein, such as, among others, “can”, “might”, “may”, “e.g.”, and the like, unless specifically stated otherwise, or otherwise understood within the context as used, is generally intended to convey that certain embodiments include, while other embodiments do not include, certain features, elements and/or states.

What is claimed is:

1. A sensor assembly for sensing fluid pressure in a surgical cassette, the sensor assembly comprising:
a tubing having a first portion and a second portion;
a follower assembly operable to engage with a retaining ring of the surgical cassette coupled to the first portion, the follower assembly comprising:
a follower coupled to a cup; and
a sensor component;
a collar on the second portion of the tubing operable to fit into a bearing of a pump head of a fluid pump assembly within a surgical console operable to receive the surgical cassette; and
a biasing element between the follower assembly and the collar operable to apply a biasing force to the follower assembly to engage the follower with the surgical cassette.

2. The sensor assembly of claim 1, wherein the follower assembly comprises a mechanical stop positioned between the follower and the cup, the mechanical stop being disposed at an end of the first portion of the tubing.

3. The sensor assembly of claim 2, wherein the mechanical stop comprises a first alignment mechanism corresponding to a second alignment mechanism on an inside portion of the cup.

4. The sensor assembly of claim 2, wherein the sensor component comprises an inductive sensor positioned in a center portion of the follower.

5. The sensor assembly of claim 2, wherein the follower assembly comprises a strain relief positioned between the mechanical stop and the follower.

6. The sensor assembly of claim 5, wherein the sensor component comprises a printed circuit board assembly (PCBA) comprising a coil positioned in a center portion of the follower and abutting a portion of the strain relief.

7. The sensor assembly of claim 6, comprising a PCBA support abutting a PCBA spring, wherein:
the PCBA contacts a portion of the PCBA support; and
the PCBA contacts a portion of the cup.

8. The sensor assembly of claim 2, wherein the follower comprises a lens tube.

9. The sensor assembly of claim 8, wherein the sensor component comprises a camera lens disposed on a first end of the lens tube and a camera sensor disposed on a second end of the lens tube.

10. The sensor assembly of claim 9, wherein the follower comprises an illumination lens disposed in a center portion of the follower.

11. The sensor assembly of claim 10, wherein an illumination source is disposed behind the illumination lens.

12. The sensor assembly of claim 11, wherein a PCBA contacts a portion of the camera sensor and the illumination source.

13. The sensor assembly of claim 1, wherein the follower comprises protrusions receivable in corresponding mating elements of a retainer ring of the surgical cassette.

14. The sensor assembly of claim 13, wherein the follower assembly is receivable within a center portion of a pump.

15. The sensor assembly of claim 1, wherein the sensor component comprises an eddy current sensor.
